(19) United States
(12) Patent Application Publication
(10) Pub. No.: US 2024/0327818 A1
(43) Pub. Date: Oct. 3, 2024

(54) METHODS AND DEVICES FOR ISOLATING RNA USING EPITACHOPHORESIS

(71) Applicant: Roche Sequencing Solutions, Inc., Pleasanton, CA (US)

(72) Inventors: Pantea GHEIBI, Pleasanton, CA (US); Keynttisha JEFFERSON, Dublin, CA (US)

(21) Appl. No.: 18/580,558

(22) PCT Filed: Jul. 21, 2022
(86) PCT No.: PCT/US2022/037922

§ 371 Date: Jan. 18, 2024

Related U.S. Application Data
(60) Provisional application No. 63/225,040, filed on Jul. 23, 2021.

(51) Int. Cl. C12N 15/10; C12Q 1/6806

(52) U.S. Cl. CPC C12N 15/101; C12Q 1/6806

(57) ABSTRACT

Aspects provide a method of isolating RNA from a biological sample. The method may include adding the biological sample to a first electrolyte to form a first mixture. The method may include applying a voltage difference between a first electrode and a second electrode. A gel may include a portion of a second electrolyte. The method may include flowing, using the voltage difference, the first subset of RNA molecules into one or more focused zones within the second electrolyte to the second electrode. The method may include separating the second subset of RNA molecules from the first subset. The method may include collecting the first subset of RNA molecules by collecting a second mixture comprising the one or more focused zones. The concentration of the first subset in the second mixture is higher than the concentration of the first subset in the biological sample. Related systems are also described.

### CROSS-REFERENCE TO RELATED PATENT APPLICATIONS

The present application claims benefit of priority to U.S. Provisional Patent Application No. 63/225,040, filed Jul. 23, 2021, which is incorporated herein by reference for all purposes.

### TECHNICAL FIELD

The present disclosure relates to the field of electrophoresis for sample analysis and relates to analysis of biological samples by selective separation, detection, extraction, isolation, purification, and/or (pre-) concentration of samples, through devices and methods for epitachophoresis.

### BACKGROUND

Electrophoresis approaches have been used to separate and analyze samples for a variety of purposes, such as for identifying a particular substance or for determining the size and type of molecules in a solution. For example, a variety of molecular biology applications have employed electrophoresis to separate proteins or nucleic acids, determine molecular weight, and/or prepare samples for further analysis. In these and other applications, electrophoresis generally involves the movement of an electrically-charged substance (e.g., molecules or ions) under the influence of an electric field. This movement can facilitate the separation of a sample from other samples or substances. Once separated, the sample may readily be analyzed using an optical or other approach.

A variety of electrophoresis-based approaches typically are used in connection with different applications dependent on the particular needs of the analysis that to be performed. For example, isotachophoresis (“ITP”) is a concentration and separation technique, which leverages electrolytes with different electrophoretic mobility to focus, and in some cases separate, ionic analytes into distinct zones (“focused zones”). In ITP, analytes simultaneously focus and separate between high effective mobility leading electrolyte (“LE”) ions and low effective mobility trailing electrolyte (“TE”) ions. The balance of electromigration and diffusion at the zone boundaries in ITP typically results in sharp moving boundaries.

Conventionally, ITP is effected through use of devices and methods that feature capillary or microfluidic channel designs. Such devices and methods are capable of handling only small volumes (μl scale) of sample for analysis, which can make the analysis of biological samples difficult, such as the extraction of nucleic acids from blood and/or plasma. Epitachophoresis (ETP) methods and devices that provide these and other improvements are described herein.

### BRIEF SUMMARY

Epitachophoresis methods and systems described herein allow for efficient and improved extraction of smaller RNA molecules from a biological sample. The extraction of these small RNA molecules may involve using a gel in an electrolyte, where the gel helps separate the larger RNA from the smaller DNA. Larger RNA molecules tend to fold before entering the gel. These larger RNA molecules may then be slowed down in the gel or may be trapped in the gel, while smaller RNA molecules pass through.

Some aspects described herein provide a method of isolating RNA from a biological sample. The method may include adding the biological sample to a first electrolyte to form a first mixture. The method may include applying a voltage difference between a first electrode and a second electrode. The first electrode is disposed in the first mixture. The second electrode is disposed in a first portion of a second electrolyte. A gel may include a second portion of the second electrolyte. The first electrolyte is different from the second electrolyte. The method may include flowing, using the voltage difference, the first subset of RNA molecules in one or more focused zones within the second electrolyte to the second electrode. The method may include separating the second subset of RNA molecules from the first subset of RNA molecules by flowing first subset of RNA molecules through the gel faster than the second subset of RNA molecules. The method may include collecting the first subset of RNA molecules by collecting a second mixture comprising the one or more focused zones. The concentration of the first subset of RNA molecules in the second mixture is higher than the concentration of the first subset of RNA molecules in the biological sample. The second mixture may not include the second subset of RNA molecules.

Some aspects described herein provide a system for isolating RNA from a biological sample. The system may include an epitachophoresis device. The epitachophoresis (ETP) device may include a circular first electrode disposed at an outer edge of a circular channel. The ETP device may include a sample collection reservoir in a central region of the circular channel. The ETP device also includes a second electrode. The second electrode may be configured to be in closer electrical communication with the sample collection reservoir than the circular first electrode is with the sample collection reservoir. A first electrolyte and a gel are disposed in the circular channel. The gel may include a portion of a second electrolyte and a buffer. The first electrolyte is disposed to encircle the gel, and a polymeric portion of the gel is at least 0.7% on a mass per volume basis of the total volume of the second electrolyte, the buffer, and the gel. The system may include a power supply configured to deliver a voltage difference between the circular first electrode and the second electrode.

A better understanding of the nature and advantages of embodiments of the present disclosure may be gained with reference to the following detailed description and the accompanying drawings.

### BRIEF DESCRIPTION OF THE DRAWINGS

FIG. 1 provides a schematic representation of an exemplary device for effecting epitachophoresis.

FIG. 2A provides a schematic representation of a top view of an exemplary device for effecting epitachophoresis. In FIG. 2A, numbers 1-7 refer to the following: 1. Outer circular electrode: 2. Terminating electrolyte reservoir: 3. Leading electrolyte, optionally contained within a gel or otherwise hydrodynamically separated from the terminating electrolyte: 4. Leading electrolyte electrode/collection reservoir: 5. Central electrode: 6. Electric power supply; and 7. Boundary between leading and terminating electrolytes with sample ions focused in between; and the symbols r and d are used to represent the leading electrolyte reservoir radius and distance migrated by the LE/TE boundary, respectively.

FIG. 2B provides a schematic representation of a side view of an exemplary device for effecting epitachophoresis. In FIG. 2B, numbers 1-8 refer to the following: 1. Outer circular electrode: 2. Terminating electrolyte reservoir: 3. Leading electrolyte, optionally contained within a gel or otherwise hydrodynamically separated from the terminating electrolyte: 4. Leading electrolyte electrode/collection reservoir: 5. Center electrode: 6. Electric power supply: 7. Boundary between leading and terminating electrolytes with sample ions focused in between; and 8. Bottom support; and the symbols r and d are used to represent the leading electrolyte reservoir radius and distance migrated by the LE/TE boundary, respectively.

FIG. 3 provides a schematic representation of an exemplary device for effecting epitachophoresis.

FIG. 4 provides a schematic representation of an exemplary device for effecting epitachophoresis. In FIG. 4, the numbers 1-10 refer to the following: 1. Outer circular electrode: 2. Terminating electrolyte reservoir: 3. Leading electrolyte, optionally contained within a gel or otherwise hydrodynamically separated from the terminating electrolyte: 4. Opening to leading electrolyte/collection reservoir; 5. Center electrode; 6. Electric power supply: 7. Boundary between leading and terminating electrolytes with sample ions focused in between: 8. Bottom support: 9. Tube connecting device to a leading electrolyte reservoir; 10. Leading electrolyte reservoir.

FIG. 5 provides a schematic representation of an exemplary device for effecting epitachophoresis wherein the sample is loaded in between loading the leading and terminating electrolytes.

FIG. 6 provides a schematic representation of a device for effecting epitachophoresis and is referred to for equations described.

FIG. 7 shows a flowchart for a method for isolating RNA according to embodiments of the present invention.

FIG. 8 shows an epitachophoresis device according to embodiments of the present invention.

FIG. 9 shows size profile of short extracted RNA according to embodiments of the present invention.

FIG. 10 shows the size distribution from mapped miRNA sequences according to embodiments of the present invention.

FIG. 11 shows Venn diagrams of miRNA transcripts extracted for ETP and for RNA columns according to embodiments of the present invention.

FIG. 12 shows a scatterplot comparison of mRNA transcripts according to embodiments of the present invention.

FIG. 13 shows a graph of the relevant lung cancer miRNA and variability per extraction with ETP or an RNA column according to embodiments of the present invention.

FIG. 14 illustrates a measurement system according to embodiments of the present invention.

FIG. 15 shows a computer system according to embodiments of the present invention.

### TERMS

As used herein, the term “isotachophoresis” generally refers to the separation of charged particles by using an electric field to create boundaries or interfaces between materials (e.g., between the charged particles and other materials in a solution). ITP generally uses multiple electrolytes, where the electrophoretic mobilities of sample ions are less than that of a leading electrolyte (LE) and greater than that of a trailing electrolyte (TE) that are placed in a device for ITP. The leading electrolyte (LE) generally contains a relatively high mobility ion, and a trailing electrolyte (TE) generally contains a relatively low mobility ion. The TE and LE ions are chosen to have effective mobilities respectively lower and higher than target analyte ions of interest. That is, the effective mobility of analyte ions is higher than that of the TE and lower than that of the LE. These target analytes have the same sign of charge as the LE and TE ions (i.e., a co-ion). An applied electric field causes LE ions to move away from TE ions and TE ions to trail behind. A moving interface forms between the adjacent and contiguous TE and LE zones. This creates a region of electric field gradient (typically from the low electric field of the LE to the high electric field of the TE). Analyte ions in the TE overtake TE ions but cannot overtake LE ions and accumulate (“focus” or form a “focused zone”) at the interface between TE and LE. Alternately, target ions in the LE are overtaken by the LE ions; and also accumulate at interface. With judicious choice of LE and TE chemistry, ITP is fairly generally applicable, can be accomplished with samples initially dissolved in either or both the TE and LE electrolytes, and may not require very low electrical conductivity background electrolytes.

As used herein, the term “epitachophoresis” generally refers to methods of electrophoretic separation that are performed using a circular or spheroid and/or concentric device and/or circular and/or concentric electrode arrangement, such as by use of the circular/concentric and/or polygonal devices as described herein. Due to a circular/concentric or another polygonal arrangement that is used during epitachophoresis: unlike conventional isotachophoresis devices, the cross section area changes during migration of ions and zones, and the velocity of the zone movement is not constant in time due to the changing cross sectional area. Thus, an epitachophoretic arrangement does not strictly follow conventional isotachophoretic principles, wherein the zones migrate with constant velocities. Notwithstanding these significant differences as shown herein epitachophoresis can be used to efficiently separate and focus charged particles by using an electric field to create boundaries or interfaces between materials that may have different electrophoretic mobilities (e.g., between the charged particles and other materials in a solution). LE and TE, as described for use with ITP, can be used for epitachophoresis as well. In some embodiments, epitachophoresis may be effected using constant current, constant voltage, and/or constant power. In some embodiments, epitachophoresis may be effected using varying current, varying voltage, and/or varying power. In some embodiments, epitachophoresis may be effected within the context of devices and/or an arrangement of electrodes whose shape may be described in general as circular or spheroid, such that the basic principles of epitachophoresis may be accomplished as described herein. In some embodiments, epitachophoresis may be effected within the context of devices and/or an arrangement of electrodes whose shape may be described in general as polygons, such that the basic 20) principles of epitachophoresis may be accomplished as described herein. In some embodiments, epitachophoresis may be effected by any non-linear, contiguous arrangement of electrodes, such as electrodes arranged in the shape of a circle and/or electrodes arranged in the shape of a polygon.

As used herein, the terms “in vitro diagnostic application (IVD application)”, “in vitro diagnostic method (IVD method)”, “in vitro diagnostic assay”, and the like, generally refer to any application and/or method and/or device that may evaluate a sample for a diagnostic and/or monitoring purposes, such as identifying a disease in a subject, optionally a human subject. In some embodiments, said sample may comprise nucleic acids and/or target nucleic acids from a subject and/or from a sample, optionally further wherein said nucleic acids originated from a urine sample. In some embodiments, an epitachophoresis device may be used as an in vitro diagnostic device. In some embodiments, a target analyte that has been concentrated/enriched/isolated/purified through epitachophoresis may be used in a downstream in vitro diagnostic assay. In some embodiments, an in vitro diagnostic assay may comprise nucleic acid sequencing, e.g., DNA sequencing, e.g., RNA sequencing. In some embodiments, and IVD assay may comprise gene expression profiling. In some embodiments, an in vitro diagnostic method may be, but is not limited to being, any one or more of the following: staining, immunohistochemical staining, flow cytometry, FACS, fluorescence-activated droplet sorting, image analysis, hybridization, DASH, molecular beacons, primer extension, microarrays, CISH, FISH, fiber FISH, quantitative FISH, flow FISH, comparative genomic hybridization, blotting, Western blotting, Southern blotting, Eastern blotting, Far-Western blotting, Southwestern blotting, Northwestern blotting, and Northern blotting, enzymatic assays, ELISA, ligand binding assays, immunoprecipitation, ChIP, ChIP-seq, ChIP-ChiP, radioimmunoassays, fluorescence polarization, FRET, surface plasmon resonance, filter binding assays, affinity chromatography, immunocytochemistry, gene expression profiling, DNA profiling with PCR, DNA microarrays, serial analysis of gene expression, real-time polymerase chain reaction, differential display PCR, RNA-seq, mass spectrometry, DNA methylation detection, acoustic energy, lipidomic-based analyses, quantification of immune cells, detection of cancer-associated markers, affinity purification of specific cell types, DNA sequencing, next-generation sequencing, detection of cancer-associated fusion proteins, and detection of chemotherapy resistance-associated markers.

As used herein, the terms “leading electrolyte” and “leading ion” generally refer to ions having a higher effective electrophoretic mobility as compared to that of the sample ion of interest and/or the trailing electrolyte as used during ITP and/or epitachophoresis. In some embodiments, leading electrolytes for use with anionic epitachophoresis may include, but are not limited to including, chloride, sulphate and/or formate, buffered to desired pH with a suitable base, such as, for example, histidine, TRIS, creatinine, and the like. In some embodiments, leading electrolytes for use with cationic epitachophoresis may include, but are not limited to including, potassium, ammonium, and/or sodium with acetate or formate. In some embodiments, an increase of the concentration of the leading electrolyte may result in a proportional increase of the sample zone and a corresponding increase in electric current (power) for a given applied voltage. Typical concentrations generally may be in the 10-100 mM range: however, higher or lower concentrations may also be used.

As used herein, the terms “trailing electrolyte”, “trailing ion”, “terminating electrolyte”, and “terminating ion” generally refer to ions having a lower effective electrophoretic mobility as compared to that of the sample ion of interest and/or the leading electrolyte as used during ITP and/or epitachophoresis. In some embodiments, trailing electrolytes for use with cationic epitachophoresis may include, but are not limited to including, MES, MOPS, acetate, glutamate and other anions of weak acids and low mobility anions. In some embodiments, trailing electrolytes for use with anionic epitachophoresis may include, but are not limited to including, reaction hydroxonium ion at the moving boundary as formed by any weak acid during epitachophoresis.

As used herein, the term “focused zone(s)” generally refers to a volume of solution that comprises a component that has been concentrated (“focused”) as a result of performing epitachophoresis. A component may include a target analyte or any molecule having an ionic component affected by voltages applied in ETP. A focused zone may be collected or removed from a device, and said focused zone may comprise an enriched and/or concentrated amount of a desired sample, e.g., a target analyte, e.g., a target nucleic acid. In the epitachophoresis methods described herein the target analyte generally becomes focused in the center of the device, e.g., a circular or spheroid or other polygonal shaped device.

As used herein, the terms “band” and “ETP band” generally refer to a zone (e.g. focused zone) of ion, analyte, or sample that travels separately from other ions, analytes, or samples during electrophoretic (e.g., isotachophoretic, or epitachophoretic) migration. A focused zone within an epitachophoresis device may alternatively be referred to as an “ETP band”. In some embodiments, an ETP band may comprise one or more types of ions, analytes, and/or samples. In some instances, an ETP band may comprise a single type of analyte whose separation from other materials present in a sample is desired, e.g., separation of target nucleic acid from cellular debris. In some instances, an ETP band may contain more than one target analyte, e.g., polypeptides or nucleic acids sequences highly similar in sequence, e.g., allelic variants. In some instances, the ETP band may comprise different analytes of similar size or electrophoretic mobility. In such instances, the more than one target analyte may be separated by further ETP runs, e.g., under different conditions that promote separation of said more than one analyte, and/or said more than one analyte may be separated by other techniques known in the art for separation of analytes, such as those described herein. In some embodiments, an ETP band may be collected and optionally subject to further analysis after one or more ETP-based isolations/purifications and collections. In some embodiments, an ETP band may comprise one or more target analytes undergoing or that have undergone ETP-based isolation/purification and optionally collection, e.g., as a part of an ETP-run.

The term “sample” as used herein includes a specimen or culture (e.g., microbiological cultures) that includes or is presumed to include one or more target analytes. The term “sample” is also meant to include biological, environmental, and chemical samples, as well as any sample whose analysis is desired. A sample may include a specimen of synthetic origin. A sample may include one or more microbes from any source from which one or more microbes may be derived. A sample may include, but is not limited, to whole blood, skin, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), tissue samples, biopsy samples, urine, feces, sputum, saliva, nasal mucous, prostate fluid, semen, lymphatic fluid, bile, organs, bone marrow, tears, sweat, breast milk, breast fluid, embryonic cells and fetal cells.

The term “communicate” is used herein to indicate a structural, functional, mechanical, electrical, optical, thermal, or fluidic relation, or any combination thereof, between two or more components or elements. As such, the fact that one component is said to communicate with a second component is not intended to exclude the possibility that additional components may be present between, and/or operatively associated or engaged with, the first and the second component.

As used herein, a “subject” refers to a mammalian subject (such as a human, rodent, non-human primate, canine, bovine, ovine, equine, feline, etc.) to be treated and/or one from whom a sample is obtained.

“Detecting” a sample within the context of an epitachophoresis device, system, or machine may comprise detecting its position at one, several, or many points throughout the device. Detection may generally occur by any one or more means that do not interfere with desired device, system, or machine function and with methods performed using said device, system, or machine. In some embodiments, detection encompasses any means of electrical detection, e.g., through the detection of conductivity, resistivity, voltage, current, and the like. Furthermore, in some embodiments, detection may comprise any one or more of the following: electrical detection, thermal detection, optical detection, spectroscopic detection, photochemical detection, biochemical detection, immunochemical detection, and/or chemical detection. In some embodiments, one or more RNA molecules may be detected during ETP-based isolation/purification and optionally collection of said one or more RNA molecules. Moreover, sample detection within the context of ETP devices and methods of ETP are further described in U.S. Application Ser. No. US 2020/0282392 A1; and PCT Publication No. WO 2020/074742 A1, the entire contents of all of which are incorporated herein for all purposes.

In a sample analysis device or system, the term “sample collection volume” refers to a volume of sample intended for collection, e.g., by a robotic liquid handler, during or following analysis. In a device for effecting epitachophoresis, or a system comprising such a device, the sample collection volume is the volume intended for collection that comprises sample during or following epitachophoresis. In some embodiments, the sample collection volume may be located in the central well of a device or system described herein. In some embodiments, the sample collection volume may be located anywhere that permits collection of the desired sample. In some embodiments, the sample collection volume may be anywhere between the sample loading area and the leading electrolyte electrode/collection reservoir. The sample collection volume may be comprised by any suitable area, container, well, or space of the device or system. In some embodiments, the sample collection volume is comprised by a well, membrane, compartment, vial, pipette, or the like.

As used herein, the term “ETP-based isolation/purification” generally refers to devices and methods comprising ETP, e.g., devices on which ETP may be effected, e.g., methods comprising effecting ETP, wherein ETP focuses one or more target analytes into one or more focused zones (e.g., one or more ETP bands), thereby isolating/purifying the one or more target analytes from other materials comprised by an initial sample. It is noted the terms “isolate” and “purify.” are used interchangeably. Furthermore, ETP based isolation/purification generally allows for subsequent collection of the one or more focused zones (one or more ETP bands) comprising said one or more target analytes. The degree of isolation/purification of one or more target analytes effected by one or more ETP-based isolations/purifications may be any degree or amount of isolation/purification of one or more target analytes from other materials. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more purity of said target analyte, e.g., as measured by an analytical technique to determine the composition of an ETP isolated/purified sample comprising one or more target analytes. In some embodiments, ETP-based isolation/purification of a target analyte from a sample may result in 1% or less, 1% or more, 5% or more, 10% or more, 15% or more, 20% or more 25% or more, 30% or more, 35% or more, 40% or more, 45% or more, 50% or more, 55% or more, 60% or more, 65% or more, 70% or more, 85% or more, 90% or more, 95% or more, or 99% or more of a target analyte being recovered from the original sample. In some embodiments, one or more ETP-based isolations/purifications may be effected to isolate/purify one or more target analytes, e.g., one or more nucleic acids. For example, in some instances, ETP-based isolation/purification may be effected on a sample comprising one or more target analytes to focus the one or more target analytes into one focused zone (ETP band), which substantially separates the one or more target analytes from other materials comprised in the original sample. The sample may be collected following ETP isolation/purification, and the isolated/collected sample may be further subject to another ETP-based isolation/purification. Optionally, the second ETP-based isolation-purification may be of such conditions so as to, in instances of more than one target analyte, isolate each of one or more target analytes into separate focused zones, each of which could optionally collected individually, thereby separating target analytes from one another, if desired.

As used herein, the term “mixed sample” generally refers to a sample comprising material from more than one source.

### DETAILED DESCRIPTION

Characterizing small RNA (e.g., smaller than 80 nt) is desirable. Such RNA, also termed micro RNA or miRNA, have been associated with certain disorders, including cancer. Extracting and characterizing small RNA from biological samples is desirable. Since small RNA do not represent a high percentage of the total RNA from cells and tissue (including fresh, frozen, and fixed samples), an ideal extraction system should be able to exclude the undesired RNA (large, e.g., ribosomal RNA).

Previous techniques including using RNA columns. RNA columns may be used to separate out RNA. These methods involve isolating RNA with membranes and centrifugation. Cells in a sample are lysed. The cell lysate is mixed with a binding buffer and ethanol and isopropanol. The resulting mixture is passed through the RNA column. RNA bind to a membrane. The membrane is then washed multiple times, which involves using centrifugation. The washing process is labor and time intensive. During washing, the RNA is still bound to the membrane. After washing, water is added to the membrane, and the RNA is released. The RNA is then eluted from the membrane. RNA columns extract both small and large RNA. Recovering only the small RNA may require filtering the small RNA through another technique. DNA may also be bound to the membrane if DNA is not digested or removed from a sample. In some cases, the membranes may be overloaded with RNA. The membranes used in these columns can clog or be contaminated when too much sample is used or if the sample includes DNA. These membranes can fail and need to be replaced. Automation systems for centrifugation or vacuum can be complicated and expensive. The yield for small RNA extraction may be low, as the extraction depends on multiple systems (e.g., centrifugation, membrane, vacuum, binding) working effectively together.

Epitachophoresis (ETP) provides several advantages over RNA columns and other techniques for extracting small RNA. ETP devices often involve a circular channel with concentric electrolytes. The sample is placed in the outer electrolyte. A voltage difference is applied between the central portion of the channel and the outer ring of the channel. The sample may be focused into one or more rings and moves toward the center based on charge, mass, or other properties. The focusing of the sample concentrates the sample, and the concentrated sample may be collected from the central portion. ETP does not involve moving parts for separation, and therefore mechanical failure modes are unlikely. The separation of components is driven by a voltage and not by a membrane. While ETP devices may include a membrane, the membrane is used primarily to separate an electrolyte reservoir from a collection reservoir and not for separation of the small RNA. As a result, ETP does not need to replace membranes as frequently. ETP devices may use a gel that is able to slow down the movement of large RNA. The small RNA yields for ETP devices may be higher than RNA columns. In addition, ETP devices may be able to extract more different types of small RNA compared to RNA columns.

### I. Epitachophoresis

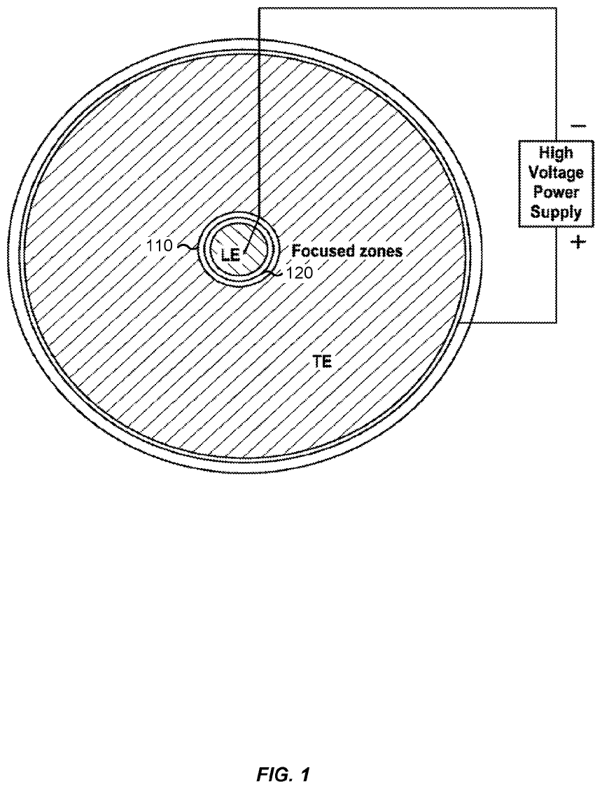

Devices for epitachophoresis generally use a concentric or polygonal disk architecture, for example, as depicted in FIG. 1-FIG. 4. Glass or ceramics may be used for fabrication of the system (i.e., material for concentric or polygonal disks) as these materials result in improved heat transfer properties that are beneficial during device operation. For example, as the flat channel of a epitachophoresis device has a favorable heat transfer capability compared to a narrow channel, over-heating (or boiling) of the focused material is generally prevented. Current/voltage programming is also suitable for adjusting the Joule heating of the device. Plastic materials may also be used for device fabrication. In general, devices are fabricated of such dimensions that accommodate a desired sample volume, such as milliliter-scale sample volumes, for example, up to 15 mL.

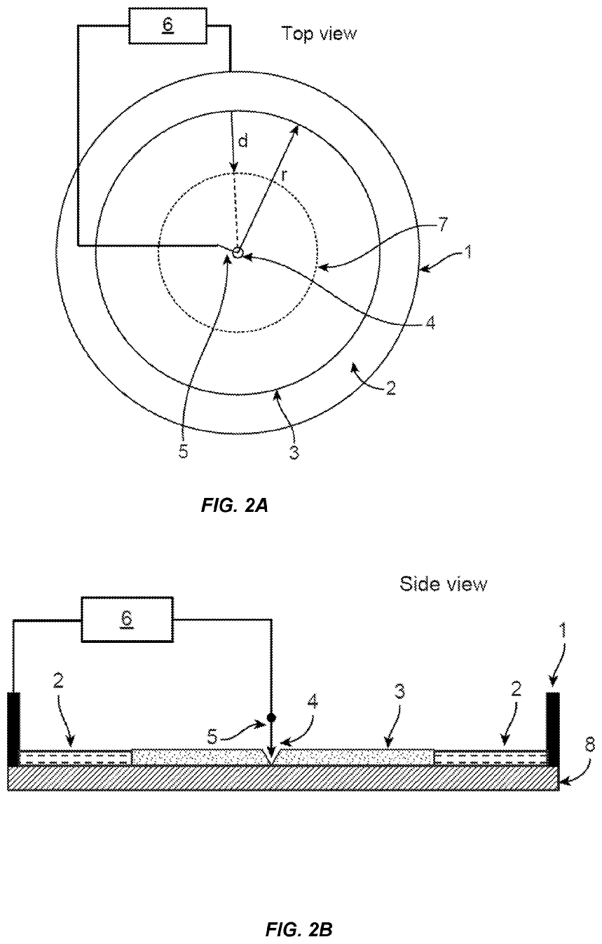

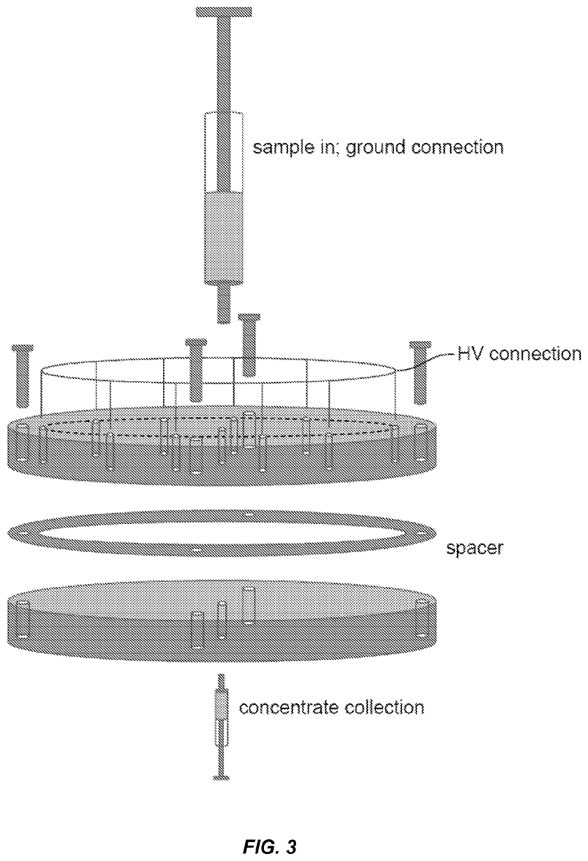

Referring to FIG. 1-FIG. 3, two concentric disks are separated by a spacer, thereby forming a flat channel for epitachophoresis sample processing. Electric current is applied through multiple high voltage connections (HV connection) and the ground connection may be in the center of the system (see FIG. 1 and FIG. 3, for example). In some instances, the sample is injected into the device through an opening in the device, e.g., in the top or the side (see, for example, FIG. 3). Application of electricity focuses the target analyte of a sample as a concentric ring that migrates to the center of the disk, and the target analyte may then be collected through a syringe at the bottom of the device (see, for example, FIG. 3). FIG. 1 shows two concentric rings, focused zone 110 and focused zone 120. Focused zone 110 and focused zone 120 may have different analytes that move at different speeds in an applied voltage as a result of their charge, mass, or other properties. As presented in FIG. 2A (top view) and FIG. 2B, an example of a device setup contains an outer circular electrode (1), terminating electrolyte (2), and leading electrolyte (3). In general, the diameter of the outer circular electrode (1) is about 10-200 mm and the diameter of the leading electrolyte ranges from a thickness (height) of about 10 μm to about 20 mm. The leading electrolyte may be stabilized by a gel, viscous additive, or otherwise hydrodynamically separated from the terminating electrolyte, such as, for example, by a membrane. The gel or hydrodynamic separation prevents mixing of the leading and terminating electrolytes during device operation. Also, in some devices mixing is prevented by using very thin (<100 um) layers of electrolytes, as is discussed further below:

Referring to FIG. 2A-FIG. 2B, in the center of the leading electrolyte is an electrode reservoir (4) with electrode (5). The assembly of the electrodes (1, 5) and electrolytes (2, 3) is placed on a flat, electrically insulating support (8). The electrolyte reservoir (4) is used for removal of the concentrated sample solution following a separation process, such as by pipetting the sample out of the reservoir, for example. Electrode reservoir (4) is also a sample collection reservoir. Outer circular electrode (1) may be disposed at the end of a circular channel in which the leading electrolyte (3) and terminating electrolyte (2) are disposed.

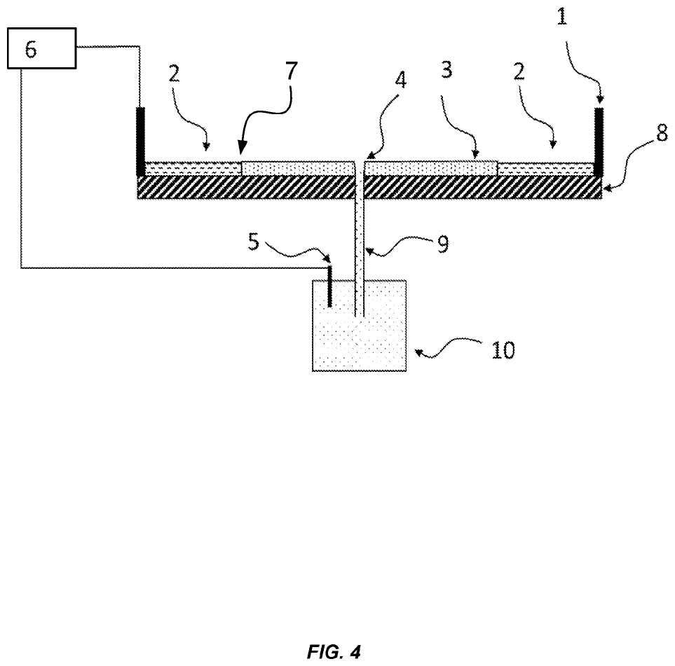

In an alternative arrangement (see FIG. 4) the center electrode (5) is moved to a leading electrolyte reservoir (10) connected with the concentrator by a tube (9). The tube (9) is connected directly or closed on one end by a semipermeable membrane (not shown). This arrangement facilitates the collection by stopping migration of large molecules according to the properties of the membrane used. This arrangement simplifies the sample collection and provides means of connecting the concentrator on-line to other devices, such as, for example, capillary analyzers, chromatography, PCR devices, enzymatic reactors, and the like. The tube (9) can also be used to supply a countercurrent flow of the leading electrolyte in an arrangement without a gel containing leading electrolyte.

In general, the gel for the leading electrolyte stabilization is formed by any uncharged material such as, for example agarose, polyacrylamide, pullulans, and the like. In some devices, the top surface is left open, or in some devices the top surface is closed, depending on the nature of the separation to be performed. If closed, the material used to cover the device is preferably a heat conducting, insulating material so as to prevent evaporation during the operation of an epitachophoresis device.

In general, the ring (circular) electrode is preferentially a gold-plated or platinum-plated stainless steel ring as this allows for maximum chemical resistance and electric field uniformity. Alternatively stainless steel and graphite electrodes may be used in some devices, particularly for disposable devices. Also, the ring (circular) electrode can be substituted with other structures that provide similar function, e.g., by an array of wire electrodes. Moreover, a 2-dimensional array of regularly spaced electrodes may additionally or alternatively be used in epitachophoresis devices. An array of regularly spaced electrodes in a circular orientation may also be used in epitachophoresis devices. Furthermore, other electrode configurations may also be used to effect different electric field shapes based on the desired sample separation (e.g., for directing the focused zones). Such configurations are described as polygon arrangements of electrodes. When divided into electrically separated segments, a switched electric field is created for time dependent shape of the driving electric field. Such an arrangement facilitates sample collection in some devices.

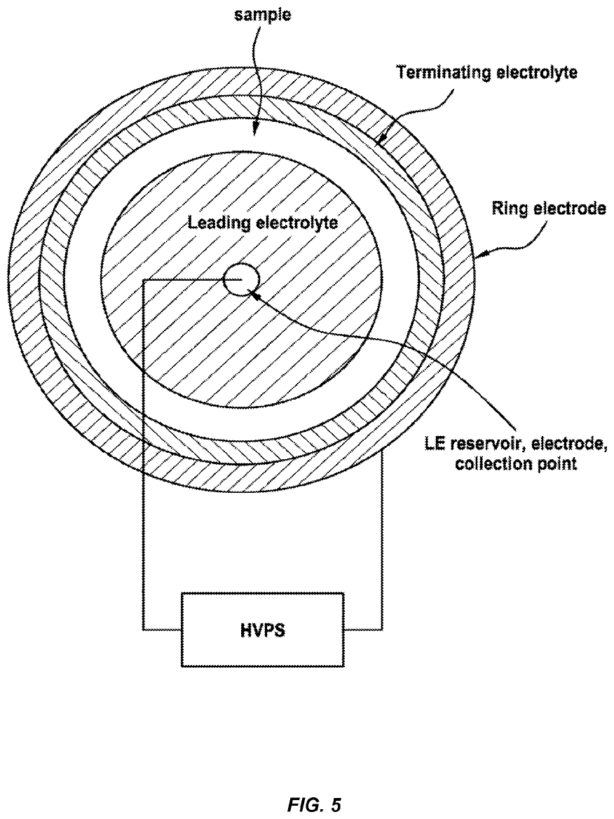

Epitachophoresis devices, such as those of the designs presented in FIG. 1-FIG. 4, may be operated in either a two electrolyte reservoir arrangement, with the leading electrolyte followed by sample mixed with terminating electrolyte or with the sample mixed with the leading electrolyte followed by the terminating electrolyte, or in a three electrolyte reservoirs arrangement, as is presented in FIG. 5. In such an arrangement, the sample may be mixed with any conducting solution. Alternatively, when the sample contains suitable terminating ions the terminating electrolyte zone can be eliminated. Referring to FIG. 2A-FIG. 2B, upon filling the terminating electrolyte (2) area with a mixture of sample and suitable terminating electrolyte and turning on the electric power supply (6), the ions start moving towards the center electrode (5) and form zones at the boundary between leading and terminating electrolytes (7). The concentrations of the sample zones during the migration adjust according to general isotachophoretic principles [Foret. F. Krivankova. L., Bocek. P . . . . Capillary Zone Electrophoresis. Electrophoresis Library. (Editor Radola. B. J.) VCH. Verlagsgesellschaft. Weinheim. 1993.], the entire contents of which are incorporated herein by reference for all purposes. Thus, the low concentrated sample ions are concentrated and highly concentrated ones are diluted. In discontinuous electrolyte systems, the concentration of a zone is regulated by the concentration of the preceding one. A discontinuous electrolyte system may include different gel structure (or presence of gel), pH value of the buffer, ionic strength of the buffer, and/or ions. Thus, the concentration in zones of minor sample components will increase, but if there is something with a high concentration (higher than the leading zone) it will get diluted. Once the sample zone enters the electrolyte reservoir (4) the separation process is stopped, and the focused material is collected in the center of the device. In practice, final concentrations of migrating zones have a concentration comparable to that of the leading ion. Typically, concentration factors of anywhere from 2 to 1,000 or even more are achieved using epitachophoresis.

In a three electrolyte reservoir arrangement, the sample is applied in between the leading and terminating electrolytes (see, for example, FIG. 5), and such an arrangement may result in slightly faster sample concentration and separation as compared to a two electrolyte reservoir arrangement.

To avoid mixing, the leading electrolyte and the trailing electrolyte may be stabilized by a neutral (uncharged) viscous media, e.g., agarose gel (see, for example, FIG. 2A-FIG. 2B, (3), which represents the leading electrolyte optionally contained within a gel or hydrodynamically separated from the terminating electrolyte).

All common electrolytes that are used for isotachophoresis can be used with the present epitachophoresis devices when the leading ions have a higher effective electrophoretic mobility than that of the sample ion(s) of interest. The opposite is true for the selected terminating ions.

The device may be operated either in positive mode (separation/concentration of cationic species) or in negative mode (separation/concentration of anionic species). The most common leading electrolytes for anionic separation using epitachophoresis include, for example, chloride, sulfate, or formate, buffered to desired pH with a suitable base, e.g., histidine, TRIS, creatinine, and the like. Concentrations of the leading electrolyte for epitachophoresis for anionic separation range from 5 mM-1 M with respect to the leading ion. Terminating ions then often include MES, MOPS, HEPES, TAPS, acetate, glutamate and other anions of weak acids and low mobility anions. Concentrations of the terminating electrolyte for epitachophoresis in positive mode range from: 5 mM-10 M with respect to the terminating ion.

For cationic separation common leading ions for epitachophoresis include, for example: potassium, ammonium or sodium with acetate or formate being the most common buffering counterions. Reaction hydroxonium ion moving boundary then serves as a universal terminating electrolyte formed by any weak acid.

In both positive and negative modes, the increase of the concentration of the leading ion results in proportional increase of the sample zone at the expense of increased electric current (power) for a given applied voltage. Typical concentrations are in the 10-100 mM range: however, higher concentrations are also possible.

Furthermore, in cases where only zone electrophoretic separation is sufficient, the device can be operated with only one background electrolyte.

Current and/or voltage programming is suitable for adjusting the migration velocity of the sample. It should be noted that in this concentric arrangement, the cross section area changes during the migration and the velocity of the zone movement is not constant in time. Thus, this arrangement does not strictly follow the isotachophoretic principle where the zones migrate with constant velocities. According to the mode of operation of the electric power supply (6) three basic cases may be distinguished: 1. Separation at Constant Current: 2. Separation at Constant Voltage; and 3. Separation at Constant Power.

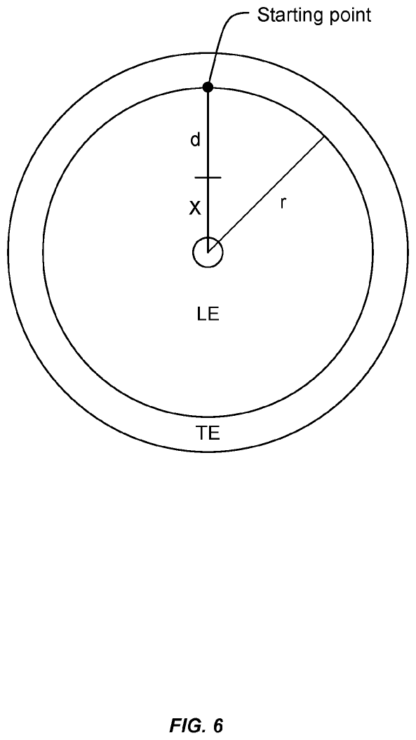

Variables for the equations described below are as follows: d=distance migrated (d<0: r>0): E=electric field strength: H=Electrolyte (gel) height: I=electric current: J=electric current density: κ=electrolyte conductivity: r=radius: S=cross-section area (area between the two electrolytes): u=electrophoretic mobility: v=velocity; and X=length from the center electrode to epitachophoresis boundary. FIG. 6 shows the relationship of the variables d, r, and X in a device.

In the common mode of operation that uses constant electric current supplied by a high voltage power supply (HVPS), the migrating zone is accelerated as it moves closer to the center due to increasing current density. With regard to separation at constant current and using a device comprising a circular architecture, e.g., a device comprising one or more circular electrodes, the relative velocity at a distance, d, depends only on the mobility (conductivity) of the leading electrolyte, as is demonstrated by the derivation of the epitachophoresis boundary velocity at v at the distance d from the start radius r as follows:

### General Equations:

$$U = IR or E = J / κ ( Ohm ’ s Law )$$

$$E = U / X ( electric field strength )$$

$$J = E κ ⇒ I = \frac{SU κ}{X} ; R = X / κ S$$

$$v = uE$$

$$S = 2 π XH$$

Epitachophoresis Boundary Velocity v at the Distance d from the Start with Radius r:

$${v}_{( d )} = {u}_{L} I / 2 π ( r - d ) h {κ}_{L} = Constant / ( r - d )$$

The ETP device may also be operated at constant voltage or constant power. The velocity of the electromigration also accelerates during the analyses performed at constant voltage and constant power.

### II. Example Methods

ETP may be used to separate smaller RNA from larger RNA in biological samples. Smaller RNA may move more quickly through the ETP channel. Larger RNA may be slowed down or stopped by gels in the ETP device.

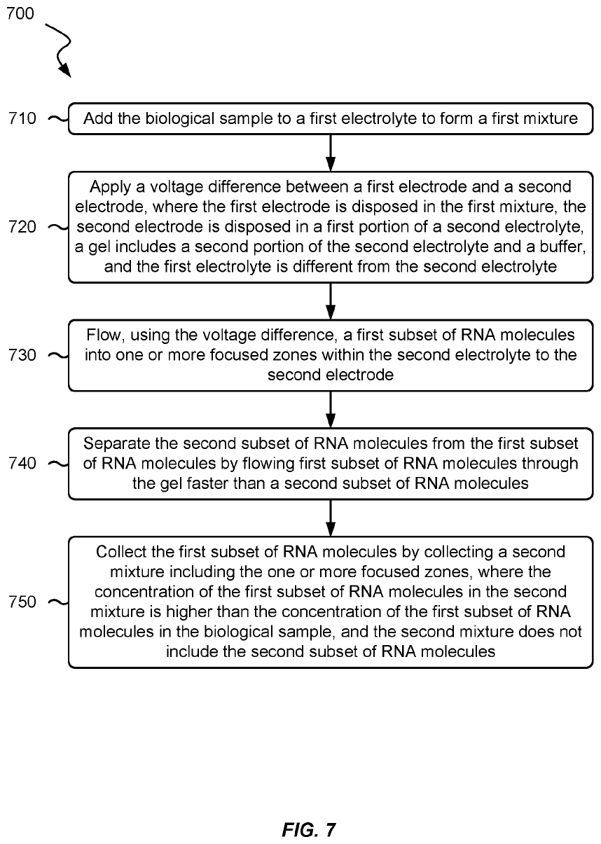

FIG. 7 is a flowchart of an example process 700 associated with methods and devices for isolating small RNA using epitachophoresis. In some implementations, one or more process blocks of FIG. 7 may be performed by a system (e.g., system 1400). In some implementations, one or more process blocks of FIG. 7 may be performed by another device or a group of devices separate from or including the system. Additionally, or alternatively, one or more process blocks of FIG. 7 may be performed by one or more components of system 1400, such as processor 1450, logic system 1430, memory 1435, external memory 1440, storage device 1445, assay device 1410, detector 1420, and/or treatment device 1460.

Frozen or fresh cells or tissue may be received. Frozen or fresh cells or tissues may be lysed to obtain a biological sample. In some embodiments, fixed cells may be received. Fixed cells may include formalin-fixed paraffin-embedded (FFPE) cells. Fixed cells may be cells prepared for pathology. Fixed cells may be cross-linked. Fixed cells may be deparaffinized, lysed, and de-crossed to obtain the biological sample.

At block 710, the biological sample may be added to a first electrolyte to form a first mixture. The biological sample may include a plurality of RNA molecules. The plurality of RNA molecules may include a first subset of RNA molecules and a second subset of RNA molecules. The first subset of RNA molecules and the second subset of RNA molecules are not separated at this point and are distributed randomly because they are obtained from the biological sample without separation. The first subset of RNA molecules may have sizes less than 80 nt, 70 nt, 60 nt, 50 nt, 40 nt, 30 nt, 25 nt, 20 nt, 15 nt, or 10 nt. The first subset of RNA molecules may be referred to as small RNA, micro RNA, or short RNA. The second subset of RNA molecules may be RNA molecules larger than the first subset of RNA molecules. In some embodiments, the second subset of RNA molecules may have sizes greater than or equal to 10 nt, 15 nt, 20 nt, 25, nt, 30 nt, 40 nt, 50 nt, 60 nt, 70 nt, 80 nt, 90 nt, 100 nt, 200 nt, 250 nt, 500 nt, or 1,000 nt. The second subset of RNA molecules may be referred to as large RNA or long RNA.

At block 720, a voltage difference is applied between a first electrode and a second electrode. First electrode may include electrode 1 of FIGS. 2A, 2B, and 4. The first electrode may be disposed in the first mixture. The second electrode may be disposed in a first portion of a second electrolyte. Second electrode may include electrode 5 in FIGS. 2A, 2B, and 4. Second electrolyte may include terminating electrolyte 2 in FIGS. 2A, 2B, and 4. The gel may include a second portion of the second electrolyte and a buffer. The gel may not extend to where the second electrode is disposed in the second electrolyte so the gel may not include the first portion of the second electrolyte. The first electrolyte is different from the second electrolyte. First electrolyte may be leading electrolyte 3 in 2A, 2B, and 4.

The gel may include a polymer such as agarose in preferred embodiments or polyacrylamide. The gel may be any gel described herein. The polymeric portion of the gel may be at least 0.7% on a mass per volume basis. The volume of the gel includes the second electrolyte and the buffer. In some embodiments, the polymeric portion of the gel may be 0.7% to 1.0%, 1.0% to 1.5%, 1.5% to 2.0%, 2.0% to 3.0%, or 3.0% or more on a mass per volume basis.

At block 730, the first subset of RNA molecules may be flowed, using the voltage difference, into one or more focused zones (e.g., bands) within the second electrolyte to the second electrode. The focused zones may be sections where the RNA (e.g., small RNA, large RNA, or total RNA) are concentrated within the first electrolyte or the second electrolyte. The first subset of RNA molecules may be concentrated into these zones as a result of the applied voltage, and the first subset of RNA molecules may move as these focused zones. The target analytes in a particular focused band may include ions with the same or similar mobility in an applied electric field. The band may be ring-shaped and be referred to as a focused zone, such as focused zones 110 and 120 in FIG. 1. The focusing may result from the applied voltage and the electrolytes. The second subset of RNA molecules may be flowed using the voltage difference.

At block 740, the second subset of RNA molecules is separated from the first subset of RNA molecules by flowing the first subset of RNA molecules through the gel faster than the second subset of RNA molecules. The difference in speed of the flow may be a result of the mass difference between the subsets. The difference in speed may also be a result of the flow through the gel. Separating the second subset of RNA molecules from the first subset of RNA molecules may include accumulating the second subset of RNA molecules within the gel. Accumulating the second subset of RNA molecules within the gel may include immobilizing the second subset of RNA molecules within the gel. The second subset of RNA molecules may include longer RNA molecules that may fold into configurations that may not pass easily through the gel. The gel may trap large RNA.

At block 750, the first subset of RNA molecules may be collected by collecting a second mixture comprising the one or more focused zones. The concentration of the first subset of RNA molecules in the second mixture is higher than the concentration of the first subset of RNA molecules in the biological sample. The second mixture does not include the second subset of RNA molecules. The concentration of the first subset of RNA molecules in the second mixture may be in the picogram or nanogram range. The first subset of RNA molecules may have an average (mean) or median length from 20 to 25 nt, 25 to 30 nt, 30 to 35 nt, 35 to 40 nt, 40 to 50 nt, 50 to 60 nt, 60 to 70 nt, or 70 to 80 nt. The ratio of small RNA molecules in the second mixture to large RNA molecules in the second mixture may be 10 to 20, 20 to 30, 30 to 40, 40 to 50, 50 to 60, 60 to 70, 70 to 80, 80 to 90, 90 to 100, 100 to 500, or 500 or more. The first subset of RNA molecules may include at least 550 unique sequences.

A membrane may separate the second electrode from the gel. Collecting the first subset of RNA molecules may include accumulating the first subset of RNA molecules on the side of the membrane with the gel. The membrane may not allow the first subset of RNA molecules to pass through toward the second electrode. The membrane may have a molecular weight cutoff of 5,000 Da, 4,000 Da, 3,000 Da, 2,000 Da, 1,000 Da, or less. In some embodiments, a third subset of RNA molecules may flow through the membrane to the first portion of the second electrolyte. The third subset of RNA molecules may be driven by the voltage difference. The third subset of RNA molecules may be smaller than the first subset of RNA molecules and may include RNA molecules having sizes less than 20 nt, 10 nt, or 5 nt.

The first subset of RNA molecules may be sequenced after being collected. RNA molecules may be sequenced by any suitable technology, including those described herein. For example, RNA may undergo an in vitro diagnostic assay.

Process 700 may include additional implementations, such as any single implementation or any combination of implementations described above and/or in connection with one or more other processes described elsewhere herein. For example, any gel concentration percentage may be used with any membrane MWCO.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

### III. Example ETP Devices

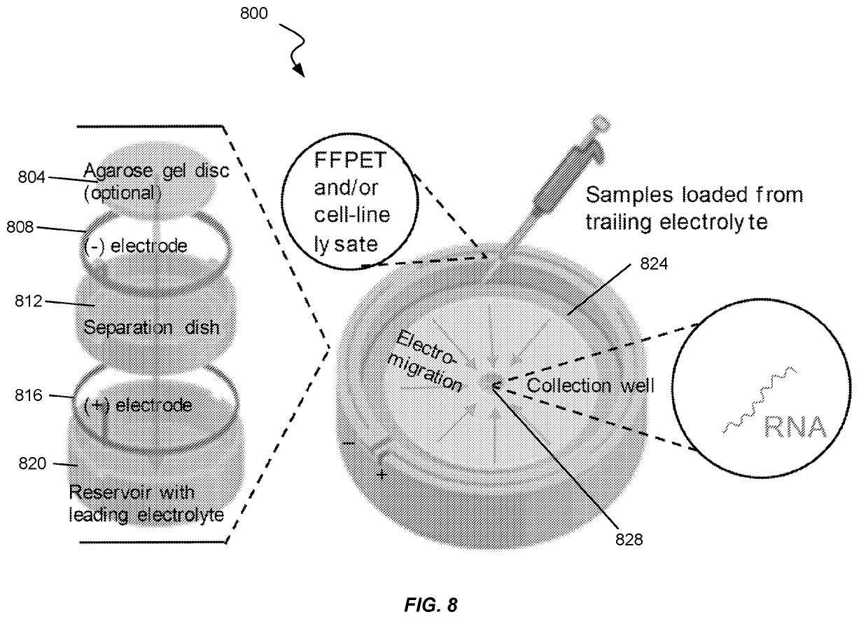

Systems for isolating small RNA from a biological sample may include an epitachophoresis device. FIG. 8 shows an example of an ETP device 800. The left side of the figure shows an exploded view of ETP device 800. At the top of the device is a gel disc 804, which may contain agarose, the leading electrolyte, and the buffer. Gel disc 804 may be leading electrolyte 3 in FIGS. 2A, 2B, and 4. The gel may be any gel described herein. A negative electrode 808 may be a ring electrode that is concentric with gel disc 804. Negative electrode 808 may be outer circular electrode 1 in FIGS. 2A, 2B, and 4. Negative electrode 808 may be disposed within separation dish 812. Separation dish 812 separates the trailing electrolyte and sample from a reservoir 820 with the leading electrolyte. Separation dish 812 separates negative electrode 808 from positive electrode 816. Positive electrode 816 may be a ring electrode. Positive electrode 816 may be center electrode 5 of FIGS. 2A, 2B, and 4. Separation dish 812 also serves as a base upon which electromigration of ETP occurs.

A sample including small RNA may be loaded into the trailing electrolyte, which is in annular space 824 between gel disc 804 and negative electrode 808. The sample then undergoes ETP with small RNA being collected in collection well 828. Collection well 828 may be collection reservoir 4 in FIGS. 2A, 2B, and 4.

The epitachophoresis device may include a circular first electrode disposed at an outer edge of a circular channel. The first electrode may be any electrode described herein, including electrode 1 of FIGS. 2A, 2B, and 4 and negative electrode 808 of FIG. 8. The epitachophoresis device may also include a sample collection reservoir. The sample collection reservoir may be reservoir 4 in FIGS. 2A and 2B or reservoir 10 in FIG. 4 and collection well 828 of FIG. 8. The sample collection reservoir may be in a central region of the circular channel. Additionally, the epitachophoresis device may include a second electrode. The second electrode may be ring-shaped, a plate, or a rod. The second electrode may be electrode 5 in FIGS. 2A, 2B, and 4 and positive electrode 816 of FIG. 8. The epitachophoresis device may be any epitachophoresis device disclosed herein.

The second electrode may be configured to be in closer electrical communication with the sample collection reservoir (e.g., collection well 828) than the circular first electrode (e.g., negative electrode 808) is with the sample collection reservoir. Closer electrical communication may refer to the resistance being lower or the current being higher given the same voltage applied. The sample collection reservoir may be physically closer to the second electrode than the first channel is to the second electrode. When the second electrode is disposed in a liquid that contacts the cavity and the first channel, the amount of liquid between the second electrode and the sample collection reservoir is less than the amount of liquid between the first electrode and the second electrode.

A first electrolyte and a gel are disposed in the circular channel. The gel may include a portion of a second electrolyte and a buffer. The first electrolyte may be disposed to encircle the gel. The first electrolyte may be disposed radially outward of the gel. The first electrolyte may be the terminating electrolyte. Terminating electrolyte may be any terminating electrolyte described herein, including terminating electrolyte 2 in FIGS. 2A, 2B, and 4. The first electrolyte may be disposed in the circular channel with a buffer, which may be different from the buffer with the second electrolyte. The buffers may be any buffers described herein.

The second electrolyte may be the leading electrolyte. A leading electrolyte may be in the center of the circular channel or closer to the center of the circular channel than the first electrolyte is to the center of the circular channel. The second electrolyte may contact the first electrolyte. The outer edge of the second electrolyte may be a circle or on a circle, and the first electrolyte may be an annulus or the edges of the first electrolyte may trace an annulus. The second electrolyte may be any leading electrolyte described herein, including leading electrolyte 3 in FIGS. 2A, 2B, and 4.

A polymeric portion of the gel may be at least 0.7% on a mass per volume basis. The polymeric portion of the gel may include agarose or polyacrylamide. The gel may be any gel described herein.

The circular channel may be any circular channel described herein. The circular channel may include the space defined by and within electrode 1 in FIGS. 2A, 2B, and 4 and annular space 824 in FIG. 8. In embodiments, the circular channel may be a circular-shaped cavity defined by the base of the ETP device. The circular channel is in fluid communication with the sample collection reservoir. For example, components in a liquid may be able to travel from the circular channel to the sample collection reservoir within the liquid. An outer diameter of the first channel may be greater than a first outer diameter at the top of the cavity.

In some embodiments, the system may include a plurality of RNA molecules disposed in the circular channel. The plurality of RNA molecules may include a first subset of RNA molecules and a second subset of RNA molecules. The first subset of RNA molecules may be small RNA, having sizes less than any size described herein (e.g., 80 nt). The second subset of RNA may be large RNA, having sizes greater than any size described herein (e.g., 80 nt). The second subset of RNA molecules may be disposed in the gel. The first subset of RNA molecules may be disposed in the sample collection reservoir. The sample collection reservoir may contain a portion of the second electrolyte and the buffer.

The system may further include a membrane. The membrane may separate the sample collection reservoir from the second electrode. The membrane may be any membrane described herein, including a semipermeable membrane. The membrane may have a molecular weight cutoff of 2,000 Da or any molecular weight cutoff described herein. The membrane may allow particles smaller than a certain size to pass through. Components in a biological sample that are intended to be analyzed may not pass through the membrane.

The system may further include a power supply configured to deliver a voltage difference between the circular first electrode and the second electrode. In some embodiments, the system may include a computer configured to control the power supply.

The power supply may deliver a constant voltage, a constant current, or a constant power.

### IV. Example Results

Experiments were performed using ETP to extract small RNA and compared to a control using an RNA column. Results show that ETP is as effective or more effective than an RNA column in extracting small RNA.

### A. Epitachophoresis Device and Equipment

The ETP device (FIG. 8) was primarily made of polypropylene. The outer dimensions were 100×100×30 mm, and the device was fabricated by injection molding. The cathode was fabricated from stainless steel. The cathode had an outer diameter of 90 mm and a 5 mm width. The bottom dish anode was laser-cut from MinGraph Flexible Graphite Sheet with Adhesive. The anode was gently taped around the perimeter of the bottom dish and had an outer diameter of 100 mm and a width of 5 mm width. A plastic cup with a semipermeable membrane (Slide-A-Lyzer™ MINI Dialysis Units 2000 Da MWCO (Thermo Fisher Scientific)) was inserted into the central collection well equipped with 1.2×9 mm o-ring. To minimize the volume the Slide-A-Lyzer was cut in half by a razor blade creating a collection cup with a volume of less than 150 ul. Custom made polycarbonate mold for gel (75 mm OD and 5 mm ID barrier for central hole) was fabricated. Gels were covered by a plastic lid (75 mm OD, 5 mm ID central hole) made by laser cutting from gel bond film.

A Qubit 4 fluorometer (Thermo Fisher Scientific) was used for quantitative amounts of DNA and RNA from extracts. Size of extracted DNA and RNA was checked on Bioanalyzer 2100 (Agilent), Tapestation 4200 (Agilent) and Pulse Pulse Field Electrophoresis Pulse Pippin (PacBio). Extracts were processed on Roche 480 lightCycler (Roche), Illumina NextSeq 500 (Illumina), Veriti Dx (Thermofisher).

### B. ETP Separation Conditions

Electrolytes and gel were prepared. The leading electrolyte (LE) solution—was 100 mM HCl-Histidine, pH 6.25 (10.49 g of L-histidine monohydrochloride monohydrate 11 g of L-histidine in 500 mL water). The trailing electrolyte (TE) solution was 20 mM TAPS-TRIS, pH 8.30 (0.605 g of TAPS and 1.625 g of TRIS in 500 mL of water). The agarose gel was in 20 mM LE (HCl-Histidine: pH 6.25). All buffers were prepared in deionized and nuclease-free water (Fisher). A 0.5% (or 0.7%) agarose gel was prepared with 500 mg (or 700 mg) of agarose mixed with 100 ml of 20 mM HCl-Histidine LE buffer in a glass Erlenmeyer flask and heated on the hot plate till boiling while stirring. The mixture was kept at boiling for 1 min. After cooling the mixture to approximately 60° C., the solution was transferred to the round gel mold. Percentages of 0.5%-0.7% agarose gel were used for cell-line experiments.

To set up the device:
1. Top dish (e.g., separation dish 812): the dialysis cup was inserted into the central well.
2. The bottom dish (e.g., reservoir 820) was filled with 100 ml of LE.
3. The top dish was inserted into the bottom dish.
4. The central well was filled with LE to prevent air pockets.
5. The gel (e.g., gel disc 804) was placed on the top dish.
6. The gel was covered with the cover lid.
7. The biological sample was mixed with 8 ml of TE and brilliant blue (10 μl 0.1 mg/ml in water).
8. The sample/TE mixture was injected into the gap between the gel and the electrode.
9. Constant power 2 W was applied.

To detect and extract collection, the brilliant blue was used to indirectly track the nucleic acid band. Another contactless method of tracking the movement of ions was monitoring the change in voltage. Upon reaching a predetermined ideal voltage for each application, ETP was stopped and the extract was collected.

### C. Materials and Methods for Pre-Treatment, Post-Treatment, and Column Control

Sample pretreatment (cell lysing and Proteinase K treatment) was performed using 100 ul Lysis buffer, and 10 ul Proteinase K from Promega ReliaPrep FFPET Total RNA Miniprep System kit was used to pretreat the approximately 325,000 cells per extraction. The mixture was digested at 56° C. for 15 min. Lysates were then cold shocked on ice for 2 min before overnight incubation at 4° C. All samples were thawed at room temperature for at least 10 minutes prior to extraction.

Extracts post-treatment, which includes DNase treatment and RNA column clean up, was performed. Prior to downstream analysis, DNA portion of the extraction was digested by 20 minutes treatment with NEB DNase I (RNase Free) at 37° C. Total RNA was retrieved from the treated sample using NEB Monarch RNA Clean Up kit (10 μg) with 3× ethanol used alongside the binding buffer to ensure retention of the small RNA in the extracts (>6nt). This condition had three extraction replicas.

A control for small RNA (i.e., micro RNA “miRNA”) extraction was performed. The Promega ReliaPrep miRNA cell and Tissue miniprep System was used to pretreat and extract total RNA from chosen cell-lines as recommended by the manufacturer. Extracts were then further concentrated by NEB Monarch RNA Clean Up (10 μg) kit prior to small RNA library preparations. Please note that conditions used for the RNA column clean-up was tailored to keeping any RNA above 6 nt size (3× used for ethanol).

To measure the nucleic acid yield, Qubit RNA HS assay kit (Thermo Fisher Scientific) was used to measure total RNA from each extraction while Qubit microRNA assay kit (Thermo Fisher Scientific) was employed to estimate the amount of small RNA (<1000 nt) extracted per sample.

To measure RNA size range following extractions, RNA size profile was checked by Tapestation capillary gel electrophoresis (High Sensitivity RNA ScreenTape for Tapestation 4200, Agilent) and by Bioanalyzer 2100 chip gel electrophoresis (High Sensitivity RNA, Agilent).

For miRNA cell-line sequencing analysis and data analysis, total RNA was extracted from the cell-line. A mass of 500 ng was used as an input into the NebNext small RNA prep for Illumina (set 1) (New England BioLabs). Library preparation was performed according to manufacturer instructions. The purified libraries were pooled and then sequenced with a NextSeq 500 instrument using the Illumina NextSeq High Output kit v3 (300 cycles) 2×75 (Illumina).

We used the publicly available nextflow pipeline to process the miRNA sequencing data. (github.com/nf-core/smrnaseq). The raw sequencing data and the normalized_CPM (counts per million) values for each of the samples is available at ncbi.nlm.nih.gov/geo/.

D. ETP Comparison with Column

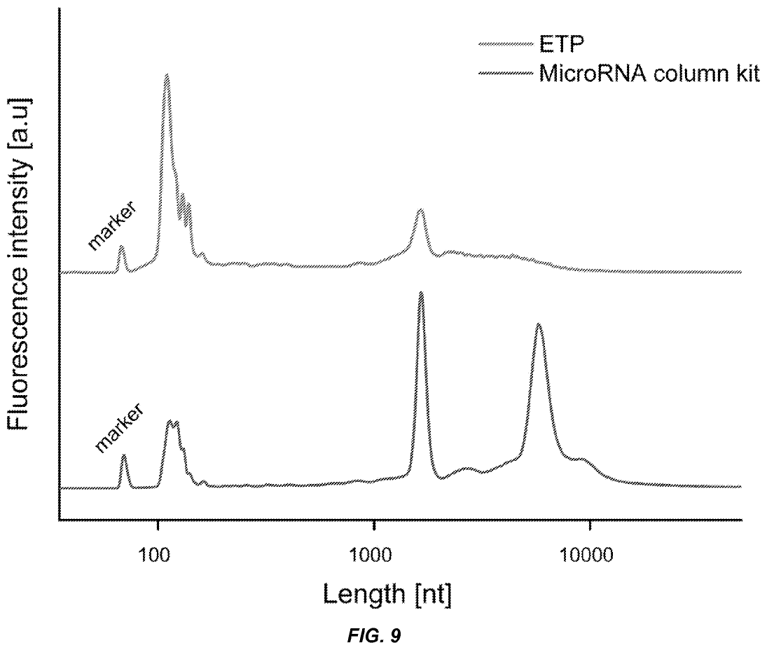

FIG. 9 shows the size profile of short extracted RNA from an electropherogram. The x-axis shows the size in nucleotides. The y-axis shows the fluorescence intensity in arbitrary units. The top line (red line) shows the results from ETP. The bottom line (black line) shows the results from the control, which is from an RNA column (ReliaPrep miRNA Cell and Tissue Miniprep System kit). The graph shows a higher amount of small RNA and exclusion of large RNA (mRNA [messenger RNA] and rRNA [ribosomal RNA]) (e.g., lengths over 1,000 nt) with ETP compared to the control.

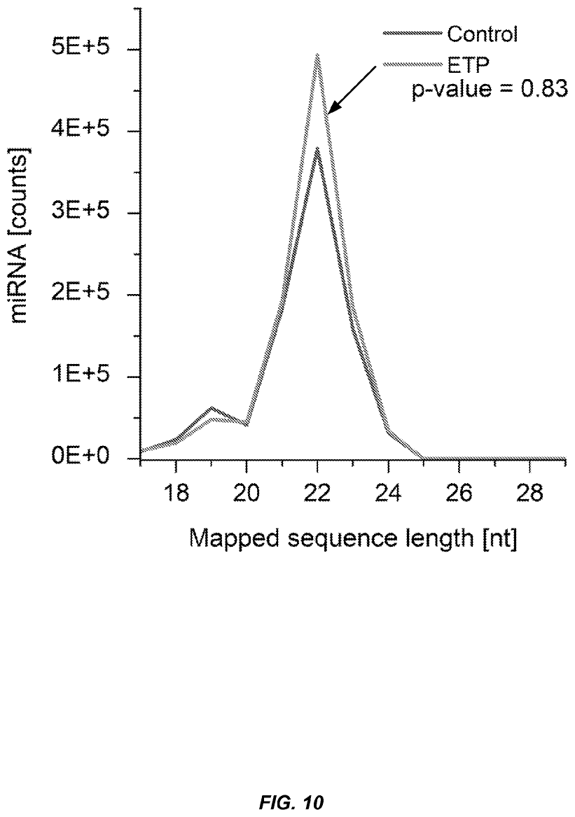

FIG. 10 shows the size distribution from mapped miRNA sequences. The x-axis shows the mapped sequence length in nucleotides. The y-axis shows amount of miRNA in counts. Sequencing libraries were subsampled to 20 million read pairs. The average length of sequences mapped to mature miRNAs was between 20-25 nt, which matches the typical length of miRNAs reported in the literature (MicroRNAs: Synthesis, mechanism, function, and recent clinical trials, Biochimica et Biophysica Acta (BBA)-Molecular Cell Research, Volume 1803, Issue 11, pp. 1231-1243).

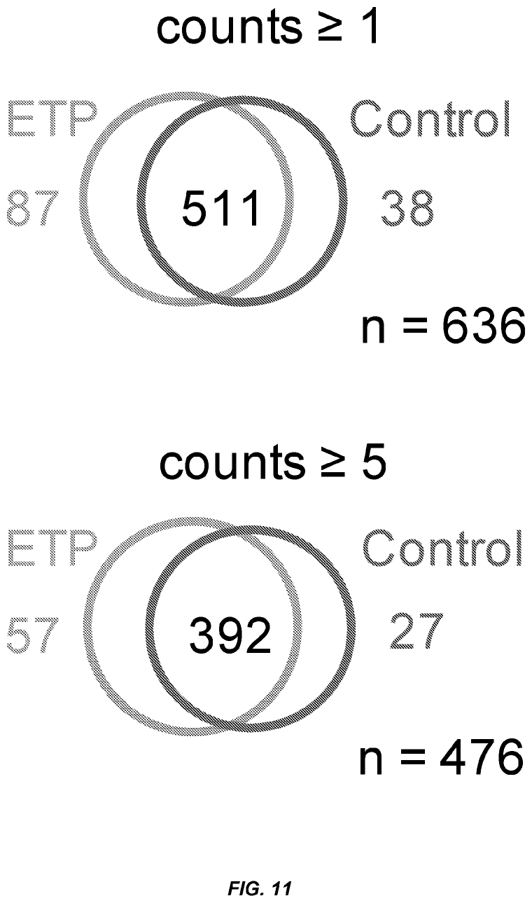

FIG. 11 shows Venn diagrams of miRNA transcripts extracted for ETP and for RNA columns. Only miRNA transcripts with abundance (greater than or equal to a predefined detection threshold of 1 count or 5 counts) in all the ETP replicas or in all column replicas are considered to be extracted by the respective method. The left circle of the Venn diagrams show the results for ETP, and the right circle shows results for RNA columns. The number of overlapping transcripts are labeled in the middle. For a threshold count greater than or equal to 1, we found 511 miRNA transcripts in both methods while ETP had 87 other unique transcripts in comparison to control which had 38. If a higher threshold applied (counts ≥5), both methods extracted 392 transcripts in common while unique transcripts for ETP were reduced to 57 and control to 27. At all those detection thresholds, ETP recovered more miRNA transcript types than the column-based method control.

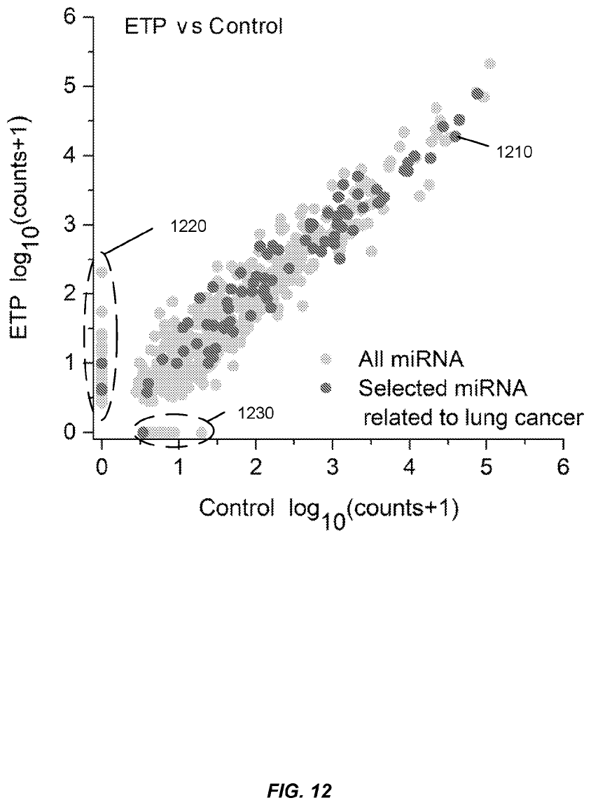

FIG. 12 shows a scatterplot comparison of miRNA transcripts having sequences known to be related to lung cancer detected with ETP and RNA columns as control. ETP is shown on the y-axis with units of log₁₀ (counts+1), and the RNA column as control is shown on the x-axis with units of log₁₀ (counts+1). Selected miRNAs related to lung cancer were detected and are shown highlighted in green (e.g., data point 1210) (Wu 2019 ncbi.nlm.nih.gov/pmc/articles/PMC6480472/.). The data points include the average values for six independent runs. Zero counts were assigned to miRNAs not found in all replicates even if they were found in some of the libraries. ETP is concordant to control and detects NSCLC miRNA across multiple logs. Nucleic acid extraction is a crucial step for next generation sequencing (NGS) and dropouts (assigned zero counts) generally decrease assay sensitivity. ETP extracts performed fewer dropouts than control, which makes ETP a suitable sample preparation method prior to NGS. FIG. 12 shows a greater number of miRNA that have zero counts for the control but a non-zero count for ETP (group 1220) than miRNA that have zero counts for ETP but a non-zero count for control (group 1230).

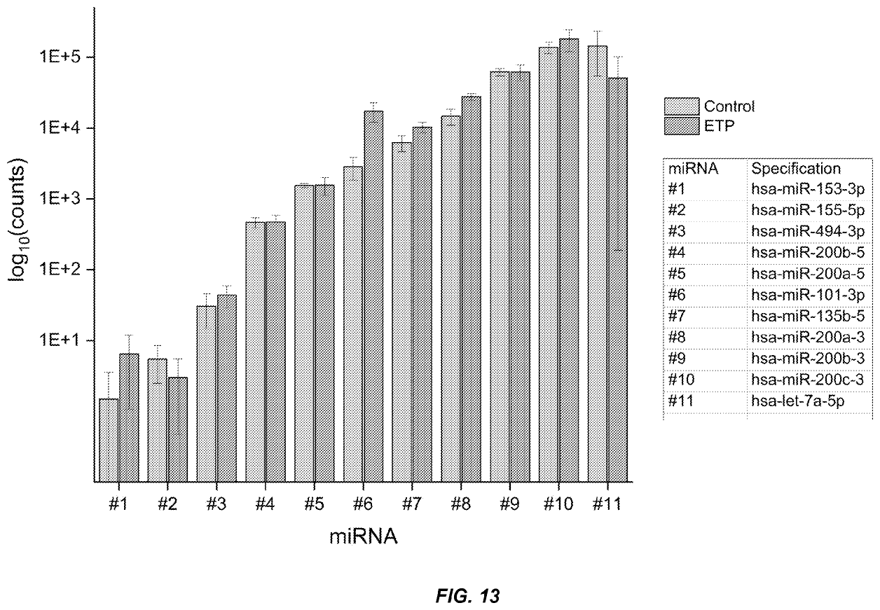

FIG. 13 shows a graph of the relevant lung cancer miRNA and variability per extraction. The x-axis lists miRNA. The y-axis shows the count on a logarithmic scale. The graph shows that for most miRNA, there is no significant difference between counts obtained from extraction by ETP (red, right column in each set) or by RNA column (gray, left column in each set). This shows ETP does not filter out or lose certain miRNA more than the control. Most miRNA transcripts showed higher counts with ETP. Three transcripts (hsa-miR-101-3p, hsa-miR-135b-5p, and hsa-miR-200a-3p) showed that ETP resulted in a significantly higher count than with an RNA column.

FIGS. 9 to 13 show that ETP is effective at extracting miRNA for further analysis. Additionally, data show that ETP can extract more miRNA transcripts and higher amounts a transcript than an RNA column.

### V. Example Measurement Systems

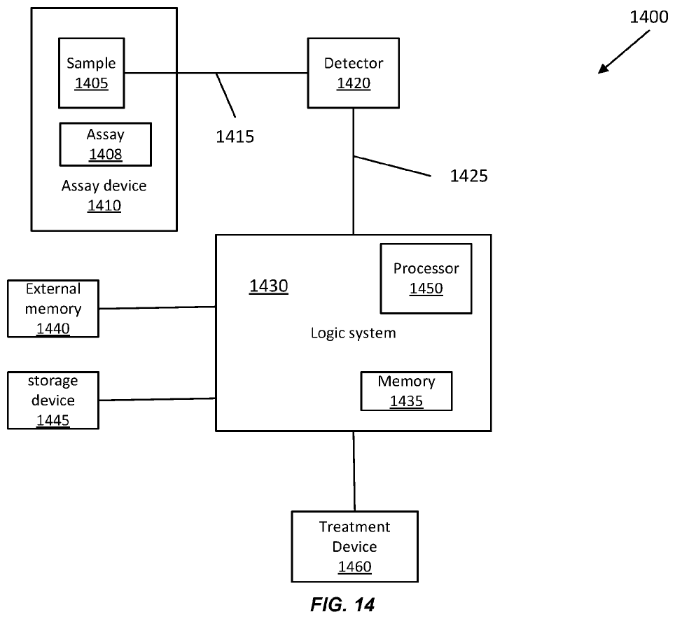

FIG. 14 illustrates a measurement system 1400 according to an embodiment of the present disclosure. The system as shown includes a sample 1405, such as cell-free DNA molecules within an assay device 1410, where an assay 1408 can be performed on sample 1405. For example, sample 1405 can be contacted with reagents of assay 1408 to provide a signal of a physical characteristic 1415. An example of an assay device can be a flow cell that includes probes and/or primers of an assay or a tube through which a droplet moves (with the droplet including the assay). Assay device 1410 may include multiple modules, including any epitachophoresis (ETP) device described herein. The ETP device can concentrate or separate sample 1405, and that concentrated sample may be sent to another module in the assay device. The other module may perform an in vitro diagnostic assay.

Physical characteristic 1415 (e.g., a fluorescence intensity, a voltage, or a current), from the sample is detected by detector 1420. Detector 1420 can take a measurement at intervals (e.g., periodic intervals) to obtain data points that make up a data signal. In one embodiment, an analog-to-digital converter converts an analog signal from the detector into digital form at a plurality of times. Assay device 1410 and detector 1420 can form an assay system, e.g., a sequencing system that performs sequencing according to embodiments described herein. A data signal 1425 is sent from detector 1420 to logic system 1430. As an example, data signal 1425 can be used to determine sequences and/or locations in a reference genome of DNA molecules. Data signal 1425 can include various measurements made at a same time, e.g., different colors of fluorescent dyes or different electrical signals for different molecule of sample 1405, and thus data signal 1425 can correspond to multiple signals. Data signal 1425 may be stored in a local memory 1435, an external memory 1440, or a storage device 1445.

Logic system 1430 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1430 and the other components may be part of a stand-alone or network connected computer system, or they may be directly attached to or incorporated in a device (e.g., a sequencing device) that includes detector 1420 and/or assay device 1410. Logic system 1430 may also include software that executes in a processor 1450. Logic system 1430 may include a computer readable medium storing instructions for controlling measurement system 1400 to perform any of the methods described herein. For example, logic system 1430 can provide commands to a system that includes assay device 1410 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Moreover, in some embodiments, the ETP device may be used with liquid handling robots that may optionally be used to effect downstream analysis of a sample that may have been focused and/or collected from said device.

Measurement system 1400 may also include a treatment device 1460, which can provide a treatment to the subject. Treatment device 1460 can determine a treatment and/or be used to perform a treatment. Examples of such treatment can include surgery, radiation therapy, chemotherapy, immunotherapy, targeted therapy, hormone therapy, and stem cell transplant. Logic system 1430 may be connected to treatment device 1460, e.g., to provide results of a method described herein. The treatment device may receive inputs from other devices, such as an imaging device and user inputs (e.g., to control the treatment, such as controls over a robotic system).

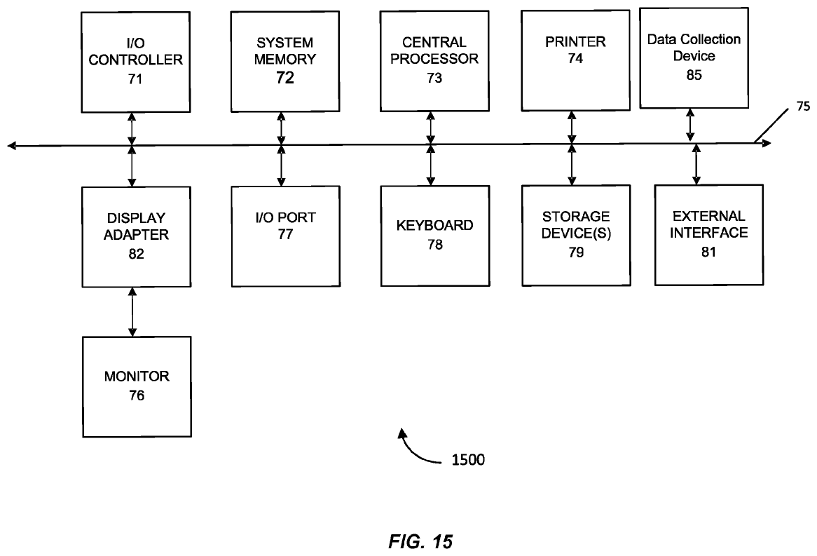

Any of the computer systems mentioned herein may utilize any suitable number of subsystems. Examples of such subsystems are shown in FIG. 15 in computer system 1500. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 15 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning). For example, I/O port 77 or external interface 81 (e.g. Ethernet, Wi-Fi, etc.) can be used to connect computer system 1500 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

What is claimed is:

1. A method of isolating RNA from a biological sample, the biological sample comprising a plurality of RNA molecules, the plurality of RNA molecules comprising a first subset of RNA molecules and a second subset of RNA molecules, the first subset of RNA molecules having sizes less than 80 nt, the second subset of RNA molecules having sizes greater than or equal to 80 nt, the method comprising:
adding the biological sample to a first electrolyte to form a first mixture;
applying a voltage difference between a first electrode and a second electrode, wherein:
the first electrode is disposed in the first mixture,
the second electrode is disposed in a first portion of a second electrolyte,
a gel includes a second portion of the second electrolyte and a buffer, and
the first electrolyte is different from the second electrolyte:
flowing, using the voltage difference, the first subset of RNA molecules into one or more focused zones within the second electrolyte to the second electrode;
separating the second subset of RNA molecules from the first subset of RNA 17 molecules by flowing the first subset of RNA molecules through the gel faster than the second subset of RNA molecules; and
collecting the first subset of RNA molecules by collecting a second mixture comprising the one or more focused zones, wherein:
the concentration of the first subset of RNA molecules in the second mixture is higher than the concentration of the first subset of RNA molecules in the biological sample, and
the second mixture does not comprise the second subset of RNA molecules.

2. The method of claim 1, wherein the first subset of RNA molecules have sizes less than 25 nt.

3. The method of claim 1, wherein a polymeric portion of the gel is at least 0.7% on a mass per volume basis.

4. The method of claim 3, wherein the polymeric portion of the gel comprises agarose.

5. The method of claim 1, wherein separating the second subset of RNA molecules from the first subset of RNA molecules comprises accumulating the second subset of RNA molecules within the gel.

6. The method of claim 5, wherein accumulating the second subset of RNA molecules within the gel comprises immobilizing the second subset of RNA molecules within the gel.

7. The method of claim 1, wherein:
a membrane separates the second electrode from the gel, and
collecting the first subset of RNA molecules comprises accumulating the first subset of RNA molecules on the side of the membrane with the gel.

8. The method of claim 7, wherein the membrane has a molecular weight cutoff of 2,000 Da or less.

9. The method of claim 7, further comprising flowing, using the voltage difference, a third subset of RNA molecules through the membrane to the first portion of the second electrolyte, wherein the third subset of RNA molecules comprises RNA molecules having sizes less than 10 nt.

10. The method of claim 1, further comprising:
receiving frozen or fresh cells, and
lysing the frozen or fresh cells to obtain the biological sample.

11. The method of claim 1, further comprising:
receiving fixed cells, and
de-crossing the fixed cells to obtain the biological sample.

12. The method of claim 1, further comprising sequencing the first subset of RNA molecules.

13. The method of claim 1, wherein the first subset of RNA molecules has an average length from 20 to 25 nt.

14. The method of claim 1, wherein the ratio of RNA molecules having sizes less than 80 nt in the second mixture to the ratio of RNA molecules having sizes greater than or equal to 80 nt in the second mixture is 100 or more.

15. The method of claim 1, wherein the first subset of RNA molecules comprises at least 550 unique sequences.

16. A system comprising:
an epitachophoresis device, the epitachophoresis device comprising:
a circular first electrode disposed at an outer edge of a circular channel,
a sample collection reservoir in the center of the circular channel, and
a second electrode, the second electrode configured to be in closer electrical communication with the sample collection reservoir than the circular first electrode is with the sample collection reservoir, wherein:
a first electrolyte and a gel are disposed in the circular channel,
the gel comprises a portion of a second electrolyte and a buffer,
the first electrolyte is disposed to encircle the gel, and
a polymeric portion of the gel is at least 0.7% on a mass per volume basis; and
a power supply configured to deliver a voltage difference between the circular first electrode and the second electrode.

17. The system of claim 16, further comprising a plurality of RNA molecules disposed in the circular channel.

18. The system of claim 17, wherein:
the plurality of RNA molecules comprises a first subset of RNA molecules having sizes less than 80 nt and a second subset of RNA molecules having sizes greater than or equal to 80 nt,
the second subset of RNA molecules is disposed in the gel, and
the first subset of RNA molecules is disposed in the sample collection reservoir.

19. The system of claim 16, further comprising a membrane, wherein the membrane separates the sample collection reservoir from the second electrode.

20. The system of claim 19, wherein the membrane has a molecular weight cutoff of 2,000 Da or less.
